(19) United States
(12) Patent Application Publication
(10) Pub. No.: US 2025/0195354 A1
(43) Pub. Date: Jun. 19, 2025

(54) CONCENTRATED CLEANSING COMPOSITIONS

(71) Applicant: Conopco, Inc., d/b/a UNILEVER, Englewood Cliffs, NJ (US)

(72) Inventors: Christine BECKWITH, Meriden, CT (US); Karoline Pugnaloni DA SILVA, Bridgeport, CT (US); Leonardus Cornelis Maria van GORKON, Naugatuck, CT (US)

(73) Assignee: Conopco, Inc., d/b/a UNILEVER, Englewood Cliffs, NJ (US)

(21) Appl. No.: 18/867,064

(22) PCT Filed: Apr. 24, 2023
(86) PCT No.: PCT/EP2023/060626

§ 371 Date: Nov. 19, 2024

Related U.S. Application Data
(60) Provisional application No. 63/346,354, filed on May 27, 2022.

(30) Foreign Application Priority Data
Jul. 15, 2022 (EP) 22185207.2

(51) Int. Cl. A61K 8/46; A61K 8/41; A61K 8/44; A61K 8/73; A61K 8/81; A61K 8/892; A61Q 5/02

(52) U.S. Cl. CPC A61K 8/463; A61K 8/416; A61K 8/44; A61K 8/466; A61K 8/737; A61K 8/8152; A61K 8/892; A61Q 5/02; A61K 2800/48; A61K 2800/5424; A61K 2800/5426; A61K 2800/5428; A61K 2800/596

(57) ABSTRACT

A concentrated cleansing composition comprises: 6 to 30% by weight based on the total weight of the concentrated cleansing composition preferably 17 to 20% by weight, of an anionic surfactant; 4 to 8% by weight based on the total weight of the concentrated cleansing composition, preferably 4 to 6% by weight, of a co-surfactant comprising sodium acyl amphoacetate, sodium acyl amphopropionate, disodium acyl amphodiacetate and disodium acyl amphodipropionate, betaine, or a combination thereof; 0.05 to 2.0% by weight based on the total weight of the concentrated cleansing composition of a sensory modifying agent, wherein the sensory modifying agent comprises isohexadecane, a silicone, a cationic polymer, a polymer of ethylene oxide, or a combination thereof; and 30 to 85% by weight of water based on the total weight of the concentrated cleansing composition; wherein a pH of the concentrated cleansing composition is 3 to 6, preferably wherein the pH is 4 to 5.

### FIELD OF THE INVENTION

Disclosed herein is a concentrated cleansing composition. The concentrated cleansing composition includes an anionic surfactant, a co-surfactant, a sensory modifying agent, and water. A pH of the concentrated cleansing composition is 3 to 6.

### BACKGROUND OF THE INVENTION

Liquid based cleansing compositions, such as shampoos and body washes, are common and enjoyed by many consumers. Such compositions typically have water as the predominant ingredient, and they are often sold in plastic bottles or tubes. The compositions are conventionally formulated to have a viscosity that is customary for consumer use and easy for evacuation from the package in which they are sold.

It is often publicized that the world's oceans will soon have more plastic than fish. Given environmental concerns and the desire for consumers and conscientious companies to do more for the planet, there is a strong desire to use less plastic when selling products, including consumer products. In view of this, efforts have been made to sell product in concentrate form, and therefore, ship product that comprises less water. The difficulty with concentrates is consumers often do not like adding additional water to the concentrate and further work, like stirring, to convert the concentrate into an end usable product. As to the concentrated product, common complaints include that the product is not homogeneous after adding water and/or of undesirable viscosity.

Efforts have been disclosed for making wash compositions. In U.S. Patent Publication No. 2019/031258 A1, rheo-fluidifying concentrated foaming compositions are described.

Other efforts have been disclosed for making wash compositions. In U.S. Patent Publication No. 2018/098923 A1, personal care compositions substantially free of sulfated surfactants are described.

Still other efforts have been disclosed for making wash compositions. In U.S. Patent Publication No. 2019/282480 A1, self-thickening cleansing compositions with N-acyl acidic amino acids or salts thereof and an amphoteric surfactant are described.

U.S. Patent Publication No. 2019/0070087 A1 discloses a method of protecting the internal protein of hair from damage, comprising the steps of applying to the hair, a hair treatment composition comprising a lactone, a disaccharide, an inorganic salt and an organic acid or salt thereof, having a pH in the range of from 3 to 6.5, prior to the application of a damage insult to the hair, and a use of such a hair treatment composition, in the treatment of hair, to protect from hair damage.

EP Patent No. 3 416 613 B1 discloses sulfate-free aqueous personal care composition comprising a surfactant system comprising at least one methyl oleoyl taurate, one isethionate and one amphoteric or zwitterionic surfactant chosen from (1) amphoacetates and diamphoacetates, (2) sultaines and (3) alkylbetaines, and from about 0.2 pbw to about 15 pbw of a conditioning agent. It is also directed toward the use of such a composition for washing keratin substrates, in particular the hair or the scalp.

U.S. Pat. No. 9,308,398 B2 discloses a multiple product system regimen for providing improved benefits to hair.

It is of increasing interest to develop a concentrate that is easy to pour and that does not need to be hydrated, resulting in a consumer product that is ready to use immediately and has very desirable characteristics, including viscosity. It is also desirable to develop a concentrate that is easy to use with a refill package or in a smaller package size to reduce plastic waste.

### SUMMARY OF THE INVENTION

Disclosed in various aspects are concentrated cleansing compositions.

A concentrated cleansing composition comprises: 6 to 30% by weight based on the total weight of the concentrated cleansing composition preferably 17 to 20% by weight, of an anionic surfactant; 4 to 8% by weight based on the total weight of the concentrated cleansing composition, preferably 4 to 6% by weight, of a co-surfactant comprising sodium acyl amphoacetate, sodium acyl amphopropionate, disodium acyl amphodiacetate and disodium acyl amphodipropionate, betaine, or a combination thereof; 0.05 to 2.0% by weight based on the total weight of the concentrated cleansing composition of a sensory modifying agent, wherein the sensory modifying agent comprises isohexadecane, a silicone, a cationic polymer, a polymer of ethylene oxide, or a combination thereof; and 30 to 85% by weight of water based on the total weight of the concentrated cleansing composition; wherein a pH of the concentrated cleansing composition is 3 to 6, preferably wherein the pH is 4 to 5.

These and other features and characteristics are more particularly described below.

### DETAILED DESCRIPTION OF THE INVENTION

Disclosed herein is a concentrated cleansing composition (e.g., a shampoo). The concentrated cleansing composition comprises an anionic surfactant, a co-surfactant, a sensory modifying agent, and water. A pH of the concentrated cleansing composition is 3 to 6, preferably, 4 to 5, or even more preferably, 4 to 4.8. The concentrated cleansing composition can be used directly from the container in which it is packaged without adding hydration. Not needing to add hydration saves the end user or consumer the added step of having to re-hydrate the product with the correct amount of hydration and mixing sufficiently to ensure homogeneity. The present cleansing composition is concentrated at a level of up to 2.5 times as compared to a base composition that is not concentrated. For example, the concentrated cleansing composition can be concentrated at a level of 2.0 times as compared to a base composition that is not concentrated, or even 1.5 times as compared to a base composition that is not hydrated. Cleansing compositions containing high levels of surfactant are typically more viscous, due to the high levels of solid present. Such high levels of surfactant generally result in more viscous compositions that are harder to pump or expel and more difficult to process, especially in large scale batches.

Typically, the phase structure of a regular, non-concentrated cleansing composition such as a shampoo composition is in the isotropic phase. However, in providing a concentrated shampoo, as the surfactant concentration is increased, the typical rheology moves from the isotropic phase to the hexagonal phase. The hexagonal phase provides a composition which is too viscous to be suitable for the typical consumer and can only be diluted very slowly.

Surprisingly, the concentrated cleansing composition disclosed herein has a viscosity that allows it to be processed in large scale packages, has flowable rheology in production and filling lines, and can be to be packaged in the same type of containers used for non-concentrated compositions. It was unexpectedly found that parity performance with current non-concentrated cleansing compositions can be achieved with the present concentrated cleansing composition.

The concentrated cleansing composition is in the isotropic phase.

Since the concentrated cleansing composition does not have to be re-hydrated, a smaller dose of the concentrated cleansing composition can be used by the end user. For example, a 2.5 times concentrated cleansing composition would have a 2.5 times smaller dose, a 2.0 times concentrated cleansing composition would have a 2.0 times smaller dose, a 1.5 times concentrated cleansing composition would have a 1.5 times smaller dose and so on.

The concentrated cleansing composition can be a personal care cleaning composition and is preferably a shampoo, make-up wash, facial wash, hand wash or personal care liquid body wash, more preferably a shampoo or a body wash, most preferably a shampoo.

The concentrated cleansing composition is one suitable to be wiped or washed off, and preferably, washed off with water.

The concentrated cleansing composition can have a viscosity of 5,000 to 30,000 centipoise (cP) 5 to 30 Pascal·seconds (Pa·s).

Viscosity, unless noted otherwise, is taken with a Discovery HR-2 Rheometer using sand blasted plates with a 100 micron gap and a shear rate of 4 s⁻¹. Viscosity is measured at 30° C.

When making the concentrated cleansing composition, the desired ingredients can be mixed with conventional apparatus under moderate shear and atmospheric conditions, with temperature being 35 to 80° C.

The concentrated cleansing composition can contain 6 to 30% by weight based on the total weight of the concentrated cleansing composition of an anionic surfactant, including any and all ranges and values subsumed therein. For example, the concentrated cleansing composition can contain 17 to 20% by weight of an anionic surfactant, including any and all ranges and values subsumed therein based on the total weight of the concentrated cleansing composition.

As to anionic surfactants suitable for use in the concentrated cleansing composition, the anionic surfactant used can include aliphatic sulfonates, such as a primary alkane (e.g., C₈-C₂₂) sulfonate, primary alkane (e.g., C₈-C₂₂) disulfonate, C₈-C₂₂ alkene sulfonate (e.g., alpha olefin sulfonate (AOS)), C₈-C₂₂ hydroxyalkane sulfonate or alkyl glyceryl ether sulfonate (AGS); or aromatic sulfonates such as alkyl benzene sulfonate. The anionic surfactant can also be an alkyl sulfate (e.g., C₁₂-C₁₈ alkyl sulfate) or alkyl ether sulfate (including alkyl glyceryl ether sulfates). The olefin sulfate can be C₁₄ to C₁₆ olefin sulfonate, for example, sodium alpha olefin sulfonate. Among the alkyl ether sulfates are those having the formula:
RO(CH₂CH₂O)ₙSO₃M
wherein R is an alkyl or alkenyl having 8 to 18 carbons, preferably 12 to 18 carbons, n has an average value of at least 1.0, preferably less than 5, and most preferably 1 to 4, and M is a solubilizing cation such as sodium, potassium, ammonium or substituted ammonium.

The anionic can also include alkyl sulfosuccinates (including mono-and dialkyl, e.g., C₆-C₂₂ sulfosuccinates); alkyl and acyl taurates (often methyl taurates), alkyl and acyl sarcosinates, sulfoacetates, C₈-C₂₂ alkyl phosphates and phosphonates, alkyl phosphate esters and alkoxyl alkyl phosphate esters, acyl lactates, C₈-C₂₂ monoalkyl succinates and maleates, sulphoacetates, alkyl glucosides and acyl isethionates, and the like.

Sulfosuccinates can be monoalkyl sulfosuccinates having the formula:
R¹O₂CCH₂CH(SO₃M)CO₂M;
and amide-MEA sulfosuccinates of the formula:
R¹CONHCH₂CH₂O₂CCH₂CH(SO₃M)CO₂M wherein R¹ ranges from C₈-C₂₂ alkyl.

Sarcosinates are generally indicated by the formula:
R²CON(CH₃)CH₂CO₂M, wherein R² ranges from C₈-C₂₀ alkyl.

Taurates are generally identified by formula:
R³CONR⁴CH₂CH₂SO₃M
wherein R³ is a C₈-C₂₀ alkyl, R⁴ is a C₁-C₄ alkyl.

M is a solubilizing cation as previously described.

The isethionates that can be used include C₈-C₁₈ acyl isethionates (including those which have a substituted head group such as a C₁₋₄ alkyl substitution, preferably methyl substitution). These esters are prepared by a reaction between alkali metal isethionate with mixed aliphatic fatty acids having from 6 to 18 carbon atoms and an iodine value of less than 20. Often at least 75% of the mixed fatty acids have from 12 to 18 carbon atoms and up to 25% have from 6 to 10 carbon atoms.

The acyl isethionate used can be an alkoxylated isethionate such as is described in Ilardi et al., U.S. Pat. No. 5,393,466, entitled “Fatty Acid Esters of Polyalkoxylated isethonic acid; issued Feb. 28, 1995; hereby incorporated by reference in its entirety. This compound has the general formula:
R⁵C—O(O)—C(X)H—C(Y)H—(OCH₂—CH₂)ₘ—SO₃M
wherein R⁵ is an alkyl group having 8 to 18 carbons, m is an integer from 1 to 4, X and Y are each independently hydrogen or an alkyl group having 1 to 4 carbons and M is a solubilizing cation as previously described.

An anionic surfactant used can be sodium lauroyl glycinate, sodium cocoyl glycinate, sodium lauroyl glutamate, sodium cocoyl glutamate, sodium lauroyl isethionate, sodium cocoyl isethionate, sodium methyl lauroyl taurate, sodium methyl cocoyl taurate, sodium laureth sulfate, sodium pareth sulfate, alpha olefin sulfonate (AOS), or a combination thereof. Such anionic surfactants are commercially available from suppliers like Galaxy Surfactants, Clariant, Sino Lion and Innospec. Sodium cocoyl isethionate, sodium methyl lauroyl taurate, sodium lauroyl glyconate, sodium methyl lauroyl isethionate, sodium laureth sulfate, sodium pareth sulfate, alpha olefin sulfonate (AOS), or a combination thereof can be the preferred anionics suitable for use when used in the concentrated cleansing composition.

The concentrated cleansing composition can comprise 4 to 8% by weight based on the total weight of the concentrated cleansing composition of a co-surfactant, including any and all ranges and values subsumed therein. For example, the concentrated cleansing composition can comprise 4 to 6, or even 4 to 4.6% by weight of the co-surfactant, based on the total weight of the concentrated cleansing composition, including any and all ranges and values subsumed therein.

The co-surfactant can comprise amphoteric, zwitterionic, nonionic, or cationic surfactants.

Amphoteric surfactants suitable (which depending on pH can be zwitterionic) include sodium acyl amphoacetates, sodium acyl amphopropionates, disodium acyl amphodiacetates and disodium acyl amphodipropionates where the acyl (i.e., alkanoyl group) can comprise a C₇-C₁₈ alkyl portion. Illustrative examples of the amphoteric surfactants include sodium lauroamphoacetate, sodium cocoamphoacetate, sodium lauroamphoacetate, sodium cocoamphoacetate and mixtures thereof.

As to the zwitterionic surfactants, such surfactants include at least one acid group. Such an acid group can be a carboxylic or a sulphonic acid group. They often include quaternary nitrogen, and therefore, can be quaternary amino acids. They should generally include an alkyl or alkenyl group of 7 to 18 carbon atoms generally comply with an overall structural formula:
R⁶—[—C(O)—NH(CH₂)_{q}—]ᵣ—N⁺—(R⁷−)(R⁸)A-B
where R⁷ is alkyl or alkenyl of 7 to 18 carbon atoms; R⁷ and R⁸ are each independently alkyl, hydroxyalkyl or carboxyalkyl of 1 to 3 carbon atoms; q is 2 to 4; r is 0 to 1; A is alkylene of 1 to 3 carbon atoms optionally substituted with hydroxyl, and B is —CO₂— or —SO₃—.

Suitable zwitterionic surfactants for use in the present invention and within the above general formula include simple betaines of formula:
R⁶—N⁺—(R⁷)(R⁸)CH₂CO₂⁻
and amido betaines of formula:
R⁶—CONH(CH₂)ₜ—N⁺—(R⁷)(R⁸)CH₂CO₂⁻ where t is 2 or 3.

In both formulae R⁶, R⁷ and R⁸ are as defined previously. R⁶ can, in particular, be a mixture of C₁₂ and C₁₄ alkyl groups derived from coconut oil so that at least half, preferably at least three quarters of the groups R⁶ have 10 to 14 carbon atoms. R⁷ and R⁸ are preferably methyl.

A further possibility is that the zwitterionic surfactant is a sulphobetaine of formula:
R⁶—N⁺—(R⁷)(R⁸)(CH₂)₃SO₃⁻
or
R⁶—CONH(CH₂)ᵤ—N⁺—(R⁷)(R⁸)(CH₂)₃SO₃⁻
where u is 2 or 3, or variants of these in which —(CH₂)₃SO₃⁻ is replaced by
—CH₂C(OH)(H)CH₂SO₃⁻.

In these formulae, R⁶, R⁷ and R⁸ are as previously defined.

Illustrative examples of the zwitterionic surfactants include betaines like cocodimethyl carboxymethyl betaine, cocamidopropyl betaine, laurylamidopropyl betaine, cocamidopropyl hydroxysultaine, lauryl hydroxysultaine, cocamide monoethanolamine, or a combination thereof. Such surfactants are made commercially available from suppliers like Stepan Company, and it is within the scope of the concentrated cleansing composition to employ mixtures of the aforementioned surfactants. In an embodiment, the zwitterionic surfactant can be cocamidopropyl betaine.

Nonionic surfactants can optionally be used in the concentrated cleansing composition. When used, nonionic surfactants are typically used at levels as low as 0.5, 1, 1.5 or 2% by weight and at levels as high as 6, 8, 10 or 12% by weight of the concentrated cleansing composition based on the total weight of the concentrated cleansing composition, including any and all ranges and values disclosed therein. The nonionics which can be used include in particular the reaction products of compounds having a hydrophobic group and a reactive hydrogen atom, for example aliphatic alcohols, acids, amides or alkylphenols with alkylene oxides, especially ethylene oxide either alone or with propylene oxide. Specific nonionic surfactant compounds are alkyl (C₆-C₂₂) phenols ethylene oxide condensates, the condensation products of aliphatic (C₈-C₁₈) primary or secondary linear or branched alcohols with ethylene oxide, and products made by condensation of ethylene oxide with the reaction products of propylene oxide and ethylenediamine. Other nonionic surfactants include long chain tertiary amine oxides, long chain tertiary phosphine oxides, dialkyl sulphoxides, or a combination thereof.

Nonionic surfactants optionally used can include fatty acid/alcohol ethoxylates having the following structures a) HOCH₂(CH₂)ₛ(CH₂CH₂O)ᵥ H or b) HOOC(CH₂)_{c}(CH₂CH₂O)_{d} H; where s and v are each independently an integer up to18; and c and d are each independently an integer from 1 or greater. In an embodiment of the invention, s and v are each independently 6 to 18; c and d are each independently 1 to 30. Other options for nonionic surfactants include those having the formula HOOC(CH₂)ᵢ—CH═CH—(CH₂)ₖ(CH₂CH₂O)_{z} H, where i, k are each independently 5 to 15; and z is 5 to 50. In another embodiment of the invention, i and k are each independently 6 to 12; and z is 15 to 35.

The nonionic surfactant can also include a sugar amide, such as a polysaccharide amide. Specifically, the nonionic surfactant can be one of the lactobionamides described in U.S. Pat. No. 5,389,279 to Au et al., entitled “Compositions Comprising Nonionic Glycolipid Surfactants issued Feb. 14, 1995; which is hereby incorporated by reference in its entirety or it can be one of the sugar amides described in U.S. Pat. No. 5,009,814 to Kelkenberg, titled “Use of N-Poly Hydroxyalkyl Fatty Acid Amides as Thickening Agents for Liquid Aqueous Surfactant Systems” issued Apr. 23, 1991; hereby incorporated into the subject application by reference in its entirety.

In an embodiment, cationic surfactants can optionally be used in the concentrated cleansing compositions.

Tetra alkyl ammonium salts are another useful class of cationic surfactants suitable for optional use. Examples include cetyl or stearyl trimethyl ammonium chloride or bromide; hydrogenated palm or tallow trimethylammonium halides; behenyl trimethyl ammonium halides or methyl sulfates; decyl isononyl dimethyl ammonium halides; ditallow (or distearyl) dimethyl ammonium halides, and behenyl dimethyl ammonium chloride.

Still other types of cationic surfactants that can be used are the various ethoxylated quaternary amines and ester quats. Examples include PEG-5 stearyl ammonium lactate (e.g., Genamin KSL manufactured by Clariant), PEG-2 coco ammonium chloride, PEG-15 hydrogenated tallow ammonium chloride, PEG-15 stearyl ammonium chloride, dipalmitoyl ethyl methyl ammonium chloride, dipalmitoyl hydroxyethyl methyl sulfate, and stearyl amidopropyl dimethylamine lactate.

Even other useful cationic surfactants suitable for optional use include quaternized hydrolysates of silk, wheat, and keratin proteins, and it is within the scope of the concentrated cleansing compositions disclosed herein to use mixtures of the aforementioned cationic surfactants. Oat peptide is another useful additive in the concentrated conditioning compositions.

If used, cationic surfactants will make up no more than 1.0% by weight of the concentrated cleansing composition based on the total weight of the concentrated cleansing compositions. When present, they typically make up 0.01 to 0.7%, and more typically, 0.1 to 0.5% by weight of the concentrated cleansing compositions based on the total weight of the concentrated cleansing composition, including any and all ranges and values subsumed therein.

In an embodiment, the concentrated cleansing compositions will be substantially free of polymeric quaternary ammonium compounds (including salts of the same). In another embodiment, the concentrated cleansing compositions will comprise less than 0.1% by weight polymeric quaternary ammonium compounds. In yet another embodiment, concentrated cleansing compositions comprises less than 0.01% by weight polymeric quaternary ammonium compounds. In even another embodiment, the concentrated cleansing compositions are free of polymeric quaternary ammonium compounds (i.e., 0.0%).

The co-surfactant can comprise sodium acyl amphoacetate, sodium acyl amphopropionate, disodium acyl amphodiacetate, disodium acyl amphodipropionate, betaine, or a combination thereof. For example, the co-surfactant can comprise sodium lauroamphoacetate, sodium cocoamphoacetate, sodium lauroamphoacetate, sodium cocoamphoacetate, cocodimethyl carboxymethyl betaine, cocamidopropyl betaine, laurylamidopropyl betaine, cocamidopropyl hydroxysultaine, lauryl hydroxysultaine, cocamide monoethanolamine, or a combination thereof, preferably wherein the co-surfactant comprises cocamidopropyl betaine, cocamidopropyl hydroxysultaine, lauryl hydroxysultaine, cocamide monoethanolamine, or a combination thereof.

The concentrated cleansing composition comprises 0.05 to 2.5% by weight based on the total weight of the concentrated cleansing composition of a sensory modifying agent, including any and all ranges and values subsumed therein. For example, the concentrated cleansing composition comprises 0.05 to 2.5% by weight, preferably 0.05 to 1.75% by weight of the sensory modifying agent based on the total weight of the concentrated cleansing composition, including any and all ranges and values subsumed therein.

The sensory modifying agent can comprise isohexadecane, a silicone, a cationic polymer, a polymer of ethylene oxide, or a combination thereof.

The silicone sensory modifying agent can comprise dimethicone, amodimethicone, cyclomethicone, dimethiconol, dimethiconol/silsesquioxane copolymer, or a combination thereof. The silicone sensory modifying agent can be present in the concentrated cleansing composition in an amount of 0.25 to 5.0% by weight, for example, 0.5 to 3.0% by weight, for example, 0.75 to 2.5% by weight, for example, 1.0 to 1.5% by weight based on the total weight of the concentrated cleansing composition, including any and all ranges and values subsumed therein. A combination of dimethicone and amodimethicone can be used in the concentrated cleansing composition.

The sensory modifying agent can comprise a cationic polymer. Suitable cationic polymers can be homopolymers which are cationically substituted or can be formed from two or more types of monomers. The weight average (Mw) molecular weight of the polymers will generally be between 100,000 and 3 million Daltons. The polymers will have cationic nitrogen containing groups such as quaternary ammonium or protonated amino groups, or a mixture thereof. If the molecular weight of the polymer is too low, then the cleansing effect is poor. If too high, then there can be problems of high extensional viscosity leading to stringiness of the composition when it is poured.

The cationic nitrogen-containing group will generally be present as a substituent on a fraction of the total monomer units of the cationic polymer. Thus, when the polymer is not a homopolymer it can contain spacer non-cationic monomer units. The ratio of the cationic to non-cationic monomer units is selected to give polymers having a cationic charge density in the required range, which is generally from 0.2 to 3.0 meq/gm (milli-equivalents per gram). The cationic charge density of the polymer is determined via the Kjeldahl method as described in the US Pharmacopoeia under chemical tests for nitrogen determination.

Suitable cationic polymers include, for example, copolymers of vinyl monomers having cationic amine or quaternary ammonium functionalities with water soluble spacer monomers such as (meth)acrylamide, alkyl and dialkyl (meth)acrylamides, alkyl (meth)acrylate, vinyl caprolactone and vinyl pyrrolidine. The alkyl and dialkyl substituted monomers preferably have C1-C7 alkyl groups, more preferably C1-3 alkyl groups. Other suitable spacers include vinyl esters, vinyl alcohol, maleic anhydride, propylene glycol and ethylene glycol.

The cationic amines can be primary, secondary or tertiary amines, depending upon the particular species and the pH of the composition. In general, secondary and tertiary amines, especially tertiary, are preferred.

Amine substituted vinyl monomers and amines can be polymerized in the amine form and then converted to ammonium by quaternization.

The cationic polymers can comprise mixtures of monomer units derived from amine- and/or quaternary ammonium-substituted monomer and/or compatible spacer monomers. Suitable (non-limiting examples of) cationic polymers include:
cationic diallyl quaternary ammonium-containing polymers including, for example, dimethyldiallylammonium chloride homopolymer and copolymers of acrylamide and dimethyldiallylammonium chloride, referred to in the industry (CTFA) as Polyquaternium 6 and Polyquaternium 7, respectively;
mineral acid salts of amino-alkyl esters of homo- and co-polymers of unsaturated carboxylic acids having from 3 to 5 carbon atoms, (as described in U.S. Pat. No. 4,009,256);
cationic polyacrylamides (as described in International Publication No. WO 1995/22311).

Other cationic polymers that can be used include cationic polysaccharide polymers, such as cationic cellulose derivatives, cationic starch derivatives, and cationic guar gum derivatives.

Cationic polysaccharide polymers suitable for use include monomers of the formula:
A-O—[R—N⁺(R¹)(R²)(R³)X⁻],
wherein: A is an anhydroglucose residual group, such as a starch or cellulose anhydroglucose residual. R is an alkylene, oxyalkylene, polyoxyalkylene, or hydroxyalkylene group, or combination thereof. R¹, R² and R³ independently represent alkyl, aryl, alkylaryl, arylalkyl, alkoxyalkyl, or alkoxyaryl groups, each group containing up to about 18 carbon atoms. The total number of carbon atoms for each cationic moiety (i.e., the sum of carbon atoms in R¹, R² and R³) is preferably about 20 or less, and X is an anionic counterion.

Another type of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from the Amerchol Corporation, for instance under the tradename Polymer LM-200.

Other suitable cationic polysaccharide polymers include quaternary nitrogen-containing cellulose ethers (e.g., as described in U.S. Pat. No. 3,962,418), and copolymers of etherified cellulose and starch (e.g., as described in U.S. Pat. No. 3,958,581). Examples of such materials include the polymer LR and JR series from Dow, generally referred to in the industry (CTFA) as Polyquaternium 10.

A particularly suitable type of cationic polysaccharide polymer that can be used is a cationic guar gum derivative, such as guar hydroxypropyltrimethylammonium chloride (commercially available from Rhodia in their JAGUAR™ trademark series). Examples of such materials are JAGUAR™ C13S, JAGUAR™ C14 and JAGUAR C17™.

Mixtures of any of the above cationic polymers can be used.

Preferred cationic polymers include cationic polysaccharide polymers, cationic diallyl quaternary ammonium-containing polymers, mineral acid salts of amino-alkyl esters of homo- and co-polymers of unsaturated carboxylic acids having from 3 to 5 carbon atoms, cationic polyacrylamines, or a combination thereof. For example, the cationic polymer can comprise cationic cellulose derivatives, cationic starch derivatives, and cationic guar gum deriviatives, dimethyldiallylammonium chloride homopolymer and copolymers of acrylamide and dimethyldiallylammonium chloride, or a combination thereof.

Cationic polymers will generally be present in the concentrated cleansing compositions at levels of from 0.01 to 5%, preferably from 0.02 to 1%, more preferably from 0.05 to 0.8% and most preferably 0.2% by total weight of cationic polymer based on the total weight of the composition, including any and all ranges and values subsumed therein.

The sensory polymer can further comprise a polymer of ethylene oxide, for example, polyethylene oxide (PEG) such as PEG-45M commercially available from DOW as POLYOX™ WSR N-60k.

The polymer of ethylene oxide can be present in the concentrated cleansing composition in an amount of 0.01 to 0.1% by weight based on the total weight of the concentrated cleansing composition, for example, 0.02 to 0.8% by weight, for example, 0.3 to 0.05% by weight.

The concentrated cleansing composition can comprise water in an amount of 30 to 85% by weight, for example, 50 to 80% by weight, for example, 40 to 78% by weight, including any and all ranges and values subsumed therein.

Inorganic salt is an optional but often desired ingredient to aid in composition thickening. Salts that may be used include NaCl, KCl, MgCl₂, CaCl₂, combinations thereof, or the like. Typically, the inorganic salt makes up 0 to 10% by weight, and preferably, 0.001 to 12% by weight, more preferably, from 0.05 to 4.5% by weight, and even more preferably, 0.09 to 2% by weight of the concentrated cleansing composition, including any and all ranges and values subsumed therein.

Polymeric viscosity aids are an optional but often desired ingredient in the concentrated cleansing composition. Preferred polymers are those generally classified as high molecular weight ethoxylated fatty acid esters. Illustrative examples include PEG 120 methyl glucose dioleate, PEG 18 glyceryloleate/cocoate, PEG 150 pentaerythritol tetrastearate, or combinations thereof, or the like. One polymeric viscosity aid is PEG 150 pentaerythritol tetrastearate which is sold under the VERSATHIX™ name by Croda. When used, such aids make up from 0.001 to 0.8%, and preferably, from 0.01 to 0.5%, and most preferably, from 0.1 to 0.3% by weight of the concentrated cleansing composition, including any and all ranges and values subsumed therein.

Adjusters to modify/buffer the pH may be used. Such pH adjusters include triethylamine, NaOH, KOH, H₂SO₄, HCl, C₆H₈O₇ (i.e., citric acid), C₃H₆O₃ (i.e., lactic acid), or a combination thereof. The pH adjusters are added at amounts to yield the desired final pH. The pH adjusters can include various alpha-hydroxy acids or a combination thereof. For example, the pH adjusters can comprise the alpha-hydroxy acids of citric acid, glycolic acid, lactic acid, malic acid, tartaric acid, or a combination thereof. The pH values can be assessed with commercial instrumentation such as a pH meter made commercially available from Thermo Scientific®. The pH adjusters can be present in an amount of 0.1 to 1.0% by weight of the concentrated cleansing composition, including any and all ranges and values subsumed therein, for example, 0.25 to 0.75% by weight, for example, 0.5 to 0.6% by weight of the concentrated cleansing composition, including any and all ranges and values subsumed therein.

Optional skin benefit agents suitable for use in the concentrated cleansing composition are limited only to the extent that they are capable of being topically applied.

Illustrative examples of the benefit agents suitable to include in the concentrated cleansing composition are acids, like amino acids, such as arginine, lysine, ceramide, valine, histidine, or a combination thereof. Additional water soluble benefit agents suitable for use include vitamin B₂, panthenol (vitamin B₅), niacinamide (vitamin B₃), vitamin B₆, vitamin C, biotin (vitamin B₇), combinations thereof or the like. Water soluble derivatives of such vitamins may also be employed. For instance, vitamin C derivatives such as ascorbyl tetraisopalmitate, magnesium ascorbyl phosphate, or ascorbyl glycoside may be used alone or in combination with each other. Other water soluble benefit agents include 4-ethyl resorcinol, extracts like sage, aloe vera, green tea, grapeseed, thyme, chamomile, yarrow, cucumber, liquorice, rosemary leaf extract, camellia sinensis leaf extract, pomegranate extract, ginger extract, collagen, hibiscus, or a combination thereof. Water soluble sunscreens like ensulizole can also be used. Total amount of optional water soluble benefit agents (including combinations) when present in the concentrated cleansing composition can be 0.0 to 10%, preferably 0.001 to 8%, and most preferably, 0.01 to 6% by weight, based on total weight of the concentrated cleansing composition and including any and all ranges subsumed therein.

It is also within the scope of the concentrated conditioning composition to optionally include oil (i.e., non-water) soluble benefit agents. In an embodiment, the concentrated conditioning composition can be substantially free of oil, meaning less than or equal to 0.01% by weight oil, based on the total weight of the concentrated cleansing composition. In an embodiment, the concentrated cleansing composition can comprise oils in an amount of less than or equal to 2.0% by weight oil, based on the total weight of the concentrated cleansing composition. For example, the oils can be present in an amount of 0.05% to 2.0% by weight oils. Illustrative examples of the oils that can be used include triglycerides (animal and/or vegetable) such as soybean oil, sunflower seed oil, coconut oil, palm kernel oil, castor oil, rapeseed oil, palm oil, grape seed oil, shea butter, cocoa butter, caprylic/capric triglyceride, safflower oil, fish oil, or a combination thereof.

Other oils desirable for use include mineral oil, jojoba oil, isoparaffins, a C₁₂-C₁₅ alkyl benzoates, polyalphaolefins, isohexadecane, silicone oils, siliconized waxes like siliconized beeswax, argan oil, marula oil, camellia oleifera seed oil, vegetable wax, petrolatum, a combination thereof (including with those oils above) or the like.

When petrolatum is used, the same typically has a melting point range from 25° to 65° C. Examples of such petrolatum are jellies like VASELINE® petroleum jelly made commercially available from Unilever, White Petroleum USP from Calumet Penreco as well as Petrolatum G2212 and White PROTOPET® from Sonneborn.

Thus, oil soluble actives or benefit agents are solubilized in the surfactants used. The only limitation with respect to such oil soluble benefit agents are that the same are suitable to provide a benefit when topically applied.

Illustrative examples of the types of oil soluble benefit agents that can optionally be used in the concentrated cleansing composition include components like stearic acid, vitamins like Vitamin A, D, E, and K (and their oil soluble derivatives), sunscreens like ethylhexylmethoxycinnamate, bis-ethyl hexyloxyphenol methoxyphenol triazine, 2-ethylhexyl-2-cyano-3,3-diphenyl-2-propanoic acid, drometrizole trisiloxane, 3,3,5-trimethyl cyclohexyl 2-hydroxybenzoate, 2-ethylhexyl-2-hydroxybenzoate, or a combination thereof.

Other optional oil soluble benefit agents suitable for use include resorcinols like 4-hexyl resorcinol, 4-phenylethyl resorcinol, 4-cyclopentyl resorcinol, 4-cyclohexyl resorcinol 4-isopropyl resorcinol, or a combination thereof. Also, 5-substituted resorcinols like 4-cyclohexyl-5-methylbenzene-1,3-diol, 4-isopropyl-5-methylbenzene-1,3-diol, or a combination thereof or the like may be used. The 5-substituted resorcinols, and their synthesis are described in commonly assigned U.S. Patent Publication No. 2016/0000669A1.

Even other oil soluble actives suitable for use include omega-3 fatty acids, omega-6 fatty acids, climbazole, farnesol, ursolic acid, myristic acid, geranyl geraniol, oleyl betaine, cocoyl hydroxyethyl imidazoline, hexanoyl sphingosine, 12-hydroxystearic acid, petroselinic acid, conjugated linoleic acid, terpineol, thymol, ProPlex (functional fatty acid), or a combination thereof.

The optional oil soluble benefit agent can be a retinoic acid precursor. The retinoic acid precursor can be retinol, retinal, retinyl propionate, retinyl palmitate, retinyl acetate, or a combination thereof. Retinyl propionate, retinyl palmitate, or a combination thereof can be typically preferred.

Still another retinoic acid precursor is hydroxyanasatil retinoate made commercially available under the name RETEXTRA® as supplied by Molecular Design International. The same may be used in a mixture with the oil soluble actives described herein.

When optional oil soluble active is used in the concentrated cleansing composition, it typically makes up from 0.0 to 1.5%, and preferably, from 0.001 to 1.5%, and most preferably, from 0.05 to 1.2% by weight of the concentrated cleansing composition. In yet another embodiment, oil makes up from 0.1 to 0.5% by weight of the total weight of the concentrated cleansing composition, including any and all ranges subsumed therein.

Preservatives can desirably be incorporated into the concentrated cleansing composition to protect against the growth of potentially harmful microorganisms. Cosmetic chemists are familiar with appropriate preservatives and routinely choose them to satisfy the preservative challenge test and to provide product stability. Preservatives for use include hydantoin derivatives and propionate salts. Particularly preferred preservatives are methylisothiazolinone (MIT), methylchloroisothiazolinone (CIT), sodium benzoate, benzoic acid, potassium sorbate, iodopropynyl butyl carbamate, phenoxyethanol, 1,2-octanediol, hydroxyacetophenone, ethylhexylglycerine, hexylene glycol, methyl paraben, propyl paraben, imidazolidinyl urea, sodium dehydroacetate, dimethyl-dimethyl (DMDM) hydantoin, benzyl alcohol, or a combination thereof. Other preservatives include sodium dehydroacetate, chlorophenesin, decylene glycol, or a combination thereof. The preservatives should be selected having regard for the use of the composition and possible incompatibilities between the preservatives and other ingredients in the emulsion. Preservatives are preferably employed in amounts ranging from 0.01% to 2.0% by weight of the total weight of the end use composition (up to 7% by weight of total concentrated cleansing composition), including any and all ranges subsumed therein. Another preservative system can include hydroxyacetophenone alone or in a combination with other preservatives. Particularly preferred is a combination of CIT and MIT stabilized with magnesium nitrate. Such a preservative can be present in the concentrated cleansing composition in an amount of 0.01 to 1.0% by weight, for example, 0.03 to 0.9% by weight, for example, 0.6 to 0.08% by weight, including any and all ranges subsumed therein. The CIT:MIT preservative system can include as actives CIT and MIT, magnesium nitrate to stabilize, water, magnesium chloride, and acetic acid. Other preferred preservatives include sodium benzoate, benzoic acid, potassium sorbate, or a combination thereof.

Thickening agents are optionally suitable for use in the concentrated cleansing composition. Particularly useful are polysaccharides. Examples include fibers, starches, natural/synthetic gums, and cellulosics. Representative of the starches are chemically modified starches such as sodium hydroxypropyl starch phosphate, and aluminum starch octenylsuccinate. Tapioca starch is often preferred, as is maltodextrin. Suitable gums include xanthan, sclerotium, pectin, karaya, arabic, agar, guar (including Acacia senegal guar), carrageenan, alginate, or a combination thereof. Suitable cellulosics include hydroxypropyl cellulose, hydroxypropyl methylcellulose, ethylcellulose, sodium carboxy methylcellulose (cellulose gum/carboxymethyl cellulose), and cellulose (e.g., cellulose microfibrils, cellulose nanocrystals or microcrystalline cellulose). Sources of cellulose microfibrils include secondary cell wall materials (e.g., wood pulp, cotton), bacterial cellulose, and primary cell wall materials. Preferably the source of primary cell wall material is selected from parenchymal tissue from fruits, roots, bulbs, tubers, seeds, leaves, and combination thereof; more preferably is selected from citrus fruit, tomato fruit, peach fruit, pumpkin fruit, kiwi fruit, apple fruit, mango fruit, sugar beet, beet root, turnip, parsnip, maize, oat, wheat, peas, and combinations thereof; and even more preferably is selected from citrus fruit, tomato fruit, and combinations thereof. A most preferred source of primary cell wall material is parenchymal tissue from citrus fruit. Citrus fibers, such as those made available by Herbacel® as AQ Plus can also be used as source for cellulose microfibrils. The cellulose sources can be surface modified by any of the known methods including those described in Colloidal Polymer Science, Kalia et al., “Nanofibrillated cellulose: surface modification and potential applications” (2014), Vol 292, Pages 5-31.

Synthetic polymers, in addition to polymeric viscosity aids, are yet another class of effective thickening agents that can optionally be used. This category includes crosslinked polyacrylates such as the Carbomers, polyacrylamides such as SEPIGEL® 305 and taurate copolymers such as SIMULGEL® EG and ARISTOFLEX® AVC, the copolymers being identified by respective INCI nomenclature as sodium acrylate/sodium acryloyldimethyl taurate and acryloyl dimethyltaurate/vinyl pyrrolidone copolymer. Another preferred synthetic polymer suitable for thickening is an acrylate-based polymer made commercially available by Seppic and sold under the name SIMULGEL™ INS100. Calcium carbonate, fumed silica, and magnesium-aluminum-silicate can also be used.

Carbomer can also be used as a suspending agent. Carbomer can be present in an amount of 0.1 to 0.5% by weight, based on the total weight of the cleansing composition, for example, 0.2 to 0.4% by weight.

The amounts of optional thickening agent, when used, may range from 0.001 to 5%, by weight of the compositions. Maltodextrin, xanthan gum, and carboxymethyl cellulose are the often preferred optional thickening agents. In an embodiment, the thickening agent can comprise comprises sodium chloride, silica, bentonite, magnesium aluminium silicate, carbomer, cellulose, or a combination thereof.

Fragrances, fixatives, opacifiers (like titanium dioxide or glycol distearate), and chelating agents may optionally be included in the antimicrobial composition. Possible chelating agents include, but are not limited to, ethylyene diaminetetraacetic acid (EDTA), diethylenetriaminepentaacetic acid (DTPA), ethylene diamine disuccinic acid (EDDS), pentasodium diethylenetriaminepentaacetate, trisodium N-(hydroxyethyl)-ethylenediaminetracetate, an acid form of EDTA, sodium thiocyanate, trisodium salt of methylglycinediacetic acid, tetrasodium glutamate diacetate and phytic acid, preferably wherein the chelating agent is ethylene diaminetetraacetic acid (EDTA), diethylenetriaminepentaacetic acid (DTPA), ethylene diamine disuccinic acid (EDDS), or a combination thereof. Each of these substances may be present in an amount of 0.03 to 5%, preferably 0.05 to 0.09% by weight of the total weight of the concentrated cleansing composition, including any and all ranges subsumed therein.

Emulsifiers having an HLB of greater than 8 may optionally be used. Illustrative examples include Tween, 40, 60, 80, polysorbate 20, or a combination thereof. Typically, emulsifiers for water continuous systems make up from 0.3 to 2.5% by weight of the concentrated cleansing composition.

Humectants can be employed as additives in the concentrated cleansing composition to assist in moisturization when such emulsions are topically applied. These are generally polyhydric alcohol type materials. Typical polyhydric alcohols include glycerol (i.e., glycerine or glycerin), propylene glycol, dipropylene glycol, polypropylene glycol (e.g., PPG-9), polyethylene glycol, sorbitol, hydroxypropyl sorbitol, hexylene glycol, 1,3-butylene glycol, isoprene glycol, 1,2,6-hexanetriol, ethoxylated glycerol, propoxylated glycerol, or a combination thereof. Most preferred is glycerin, propylene glycol, dipropylene glycol, or a combination thereof. In an embodiment, the humectant can be propylene glycol, butylene glycol, dipropylene glycol, glycerin, triethylene glycol, erythritol, capryl glycol, hyaluronic acid, polypropylene glycol-7 proypyl heptyl ether, or a combination thereof. The amount of humectant employed can be less than or equal to 1% by weight of the total weight of the concentrated cleansing composition, for example, 0.0 to 35% by weight of the total weight of the compositions. Often, humectant makes up from 0.0 to 20%, and preferably, 0.001 to 15% by weight, more preferably, 0.5 to 1.0% by weight (most preferably, 0.75 to 12% by weight) of the total weight of the concentrated cleansing composition, including any and all ranges and values subsumed therein.

The concentrated cleansing composition can further comprise a hair repair agent. The hair repair agent can comprise glucono delta lactone, sodium gluconate, trehalose, sodium sulfate, or a combination thereof. The hair repair agent can be present in an amount of 0.3 to 3.5% by weight of the concentrated cleansing composition, for example, 0.3 to 1.2% by weight based on the total weight of the concentrated cleansing composition including any and all ranges and values subsumed therein.

The concentrated cleansing composition has a level of concentration of 2.5, for example, wherein the level of concentration is 2.0, for example, wherein the level of concentration is 1.5, as compared to a base, non-concentrated cleansing composition.

As used herein, “compositions” with no qualifier is meant to mean the concentrated cleansing composition. Hydratable, as used herein, means add and/or add and absorb water (i.e., to dilute) even to a composition that has water such as a composition that is initially 30 to 85% by weight water. Skin, as used herein, is meant to include skin on the arms (including underarms), face, feet, neck, chest, hands, legs, buttocks and scalp (including hair). Such end use composition is one suitable to be wiped or washed off, and preferably, washed off with water. The concentrated cleansing composition can be a home care cleaning composition but is preferably a shampoo, make-up wash, facial wash, hand wash or personal care liquid body wash. 1 Pa-s is equal to 1000 cps. Viscosity, unless noted otherwise, is taken with a Discovery HR-2 Rheometer using sand blasted plates with a 100 micron gap and a shear rate of 4 s⁻¹. Viscosity is measured at 30° C. The concentrated cleansing composition may, optionally, comprise medicinal or therapeutic agents, but preferably, is a wash which is cosmetic and non-therapeutic.

Except where otherwise explicitly indicated, all numbers in this description indicating amounts of material or conditions of reaction, physical properties of materials and/or use are to be understood as modified by the word “about.” All amounts are by weight of the final composition, unless otherwise specified.

It should be noted that in specifying any range of concentration or amount, any particular upper concentration can be associated with any particular lower concentration or amount as well as any subranges consumed therein. In that regard, it is noted that all ranges disclosed herein are inclusive of the endpoints, and the endpoints are independently combinable with each other (e.g., ranges of “up to 25% by weight, or, more specifically, 5% by weight to 20% by weight, in inclusive of the endpoints and all intermediate values of the ranges of 5% by weight to 25% by weight, etc.). “Combination is inclusive of blends, mixtures, alloys, reaction products, and the like. Furthermore, the terms “first”, “second”, and the like herein do not denote any order, quantity, or importance, but rather are used to distinguish one element from another. The terms “a” and “an” and “the” herein do not denote a limitation of quantity and are to be construed to cover both the singular and the plural, unless otherwise indicated herein or clearly contradicted by context. The suffix “(s)” as used herein is intended to include both the singular and the plural of the term it modifies, thereby including one or more of the term (e.g., the film(s) includes one or more films). Reference throughout the specification to “one embodiment”, “one aspect”, “another embodiment”, “another aspect”, “an embodiment”, “an aspect” and so forth means that a particular element (e.g., feature, structure, and/or characteristic) described in connection with the embodiment or aspect is included in at least one embodiment or aspect described herein and may or may not be present in other embodiments or aspects. In addition, it is to be understood that the described elements may be combined in any suitable manner in the various embodiments or aspects.

All cited patents, patent applications, and other references are incorporated herein by reference in their entirety. However, if a term in the present application contradicts or conflicts with a term in the incorporated reference, the term from the present application takes precedence over the conflicting term from the incorporated reference. While particular aspects have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications, variations, improvements, and substantial equivalents.

For the avoidance of doubt the word “comprising” is intended to mean “including” but not necessarily “consisting of” or “composed of.” In other words, the listed steps, options, or alternatives need not be exhaustive.

The disclosure of the invention as found herein is to be considered to cover all aspects as found in the claims as being multiply dependent upon each other irrespective of the fact that claims may be found without multiple dependency or redundancy. Unless otherwise specified, numerical ranges expressed in the format “from x to y” are understood to include x and y. In specifying any range of values or amounts, any particular upper value or amount can be associated with any particular lower value or amount. All percentages and ratios contained herein are calculated by weight unless otherwise indicated. The various features of the present invention referred to in individual sections above apply, as appropriate, to other sections mutatis mutandis. Consequently, features specified in one section may be combined with features specified in other sections as appropriate. Any section headings are added for convenience only and are not intended to limit the disclosure in any way.

### EXAMPLES

The following examples are merely illustrative of the cleansing bar compositions disclosed herein and are not intended to limit the scope hereof.

In the following examples, various formulations were made to determine levels of concentration for the concentrated cleansing compositions disclosed herein. Sodium pareth sulfate and ethylene glycol distearate have 1,4 dioxane in their compositions, an ingredient that will soon be regulated by the state of New York. Accordingly, the addition of those ingredients had to be monitored to ensure the contaminant of 1,4 dioxane would not be above the permitted limit.

Table 1 includes a base formulation, which contains no concentration. Viscosity for this base formulation was 3-10 Pascal-second (Pa-s) (3,000-10,000 cp).

TABLE 1
Comparative Example 1: Base Formulation
containing no concentration

| | Inclusion of |
|---|---|
| | Ingredient in |
| Ingredient | Final formula |
| Water | QS |
| Sodium C12-13 Pareth Sulfate | 10 |
| Cocamidopropyl Betaine | 1.92 |
| Carbomer | 0.4 |
| Dimethiconol | 0.75 |
| Glycol Distearate | 0.22 |
| Fragrance, Preservatives, pH modifiers, appearance, | 3-4% |
| viscosity modifiers and additional performance | |
| ingredients | |

In the formulation listed in Table 2, the surfactants were doubled, while the level of carbomer was kept the same as the base formulation, but ethylene glycol distearate was removed.

Table 2 includes a two-times (2×) concentrated conditioning composition. Viscosity of this 2×cleansing was 6-10 Pa-s (6,000-30,000 cP).

TABLE 2
Example 1: 2x Concentrated cleansing composition
formulation with 0.4% by weight carbomer

| | Inclusion of |
|---|---|
| | Ingredient in |
| Ingredient | Final formula |
| Water | QS |
| Sodium C12-13 Pareth Sulfate | 10 |
| Cocamidopropyl Betaine | 4 |
| Carbomer | 0.4 |
| Dimethiconol | 0.75 |
| Fragrance, Preservatives, pH modifiers, appearance, | 3-4% |
| viscosity modifiers and additional performance | |
| ingredients | |

Since carbomer generally requires a high amount of water to process, it was difficult to process this example as a thick paste was formed.

Two additional samples were made with 0.2% by weight carbomer (Table 3) and 0% carbomer (Table 4). It was found over a period of two weeks that the composition containing 0% carbomer separated. To confirm that 0.2% by weight carbomer would be an acceptable level, the samples were placed on an accelerated stability test, using a Lumisizer centrifuge, and compared to current formulas such as those listed in Table 1 to assess separation. The sample with 0.2% carbomer had parity with the current on market cleansing compositions.

TABLE 3
Example 2: 2x concentrated cleansing formulation
with 0.2% by weight carbomer

| | Inclusion of |
|---|---|
| | Ingredient in |
| Ingredient | Final formula |
| Water | QS |
| Sodium C12-13 Pareth Sulfate | 20 |
| Cocamidopropyl Betaine | 4 |
| Carbomer | 0.2 |
| Dimethiconol | 1.5 |
| Fragrance, Preservatives, pH modifiers, appearance, | 3-4% |
| viscosity modifiers and additional performance | |
| ingredients | |

Viscosity of this 2×cleansing composition was 6 to 30 Pa-s (6,000-30,000 cP).

This 2×concentrated cleansing composition was able to be processed successfully and has a viscosity that allows it to be processed in large scale packages, with flowable rheology in production and filling lines, and can be to be packaged in the same type of containers used for non-concentrated compositions.

TABLE 4
Example 3: 2x concentrated cleansing formulation
with 0% by weight carbomer

| | Inclusion of |
|---|---|
| | Ingredient in |
| Ingredient | Final formula |
| Water | QS |
| Sodium C12-13 Pareth Sulfate | 20 |
| Cocamidopropyl Betaine | 4 |
| Carbomer | 0 |
| Dimethiconol | 1.5 |
| Fragrance, Preservatives, pH modifiers, appearance, | 3-4% |
| viscosity modifiers and additional performance | |
| ingredients | |

This cleansing formulation was separated over time and was not able to suspend appearance modifiers such as mica.

Viscosity of this 2×cleansing composition was 6-30 Pa-s (6,000-30,000 cP).

TABLE 5
Example 4: 2x concentrated cleansing formulation with
increased Betaine and 0.2% by weight carbomer

| | Inclusion of |
|---|---|
| | Ingredient in |
| Ingredient | Final formula |
| Water | QS |
| Sodium C12-13 Pareth Sulfate | 20 |
| Cocamidopropyl Betaine | 6 |
| Carbomer | 0.2 |
| Dimethiconol | 1.5 |
| Fragrance, preservatives, pH modifiers, appearance, | 3-4% |
| viscosity modifiers and additional performance | |
| ingredients | |

Viscosity of this 2×cleansing composition was 6-30 Pa-s (6,000-30,000 cP). This 2×concentrated cleansing composition was able to be processed successfully and has a viscosity that allows it to be processed in large scale packages, with flowable rheology in production and filling lines, and can be to be packaged in the same type of containers used for non-concentrated compositions.

TABLE 6
Example 5: 2x concentrated cleansing formulation with increased
Betaine, reduced SPES and 0.2% by weight carbomer

| | Inclusion of |
|---|---|
| | Ingredient in |
| Ingredient | Final formula |
| Water | QS |
| Sodium C12-13 Pareth Sulfate | 17 |
| Cocamidopropyl Betaine | 4.6 |
| Carbomer | 0.2 |
| Dimethiconol | 1.5 |
| Fragrance, preservatives, pH modifiers, appearance, | 3-4% |
| viscosity modifiers and additional performance | |
| ingredients | |

Viscosity of this 2×cleansing composition was 6-30 Pa-s (6,000-30,000 cP). This 2×concentrated cleansing composition was able to be processed successfully and has a viscosity that allows it to be processed in large scale packages, with flowable rheology in production and filling lines, and can be to be packaged in the same type of containers used for non-concentrated compositions.

TABLE 7
Example 6: 2x concentrated cleansing formulation with an alternative
surfactant system comprising Sodium Olefin Sulfonate, Cocamidopropyl
Betaine and Cocamidopropyl Hydroxysultaine

| | Inclusion of |
|---|---|
| | Ingredient in |
| Ingredient | Final formula |
| Water | QS |
| Sodium Olefin Sulfonate | 16.78 |
| Cocamidopropyl Hydroxysultaine | 2.4 |
| Cocamidopropyl Betaine | 2.9 |
| Carbomer | 0.4 |
| Dimethiconol | 1.5 |
| Polyquaternium-7 | 0.4 |
| Fragrance, preservatives, pH modifiers, appearance, | 3-4% |
| viscosity modifiers and additional performance | |
| ingredients | |

Viscosity of this 2×cleansing composition was 6-30 Pa-s (6,000-30,000 cP). This 2×concentrated cleansing composition was able to be processed successfully and has a viscosity that allows it to be processed in large scale packages, with flowable rheology in production and filling lines, and can be to be packaged in the same type of containers used for non-concentrated compositions.

1. A concentrated cleansing composition, comprising:
17 to 20% by weight based on the total weight of the concentrated cleansing composition of an anionic surfactant;
4 to 8% by weight based on the total weight of the concentrated cleansing composition, of a co-surfactant comprising sodium acyl amphoacetate, sodium acyl amphopropionate, disodium acyl amphodiacetate, disodium acyl amphodipropionate, betaine, or a combination thereof;
0.05 to 2.0% by weight based on the total weight of the concentrated cleansing composition of a sensory modifying agent, wherein the sensory modifying agent comprises isohexadecane, a silicone, a cationic polymer, a polymer of ethylene oxide, or a combination thereof; and
a thickening agent, wherein the thickening agent comprises sodium chloride, silica, bentonite, magnesium aluminium silicate, carbomer, cellulose, or a combination thereof:
30 to 85% by weight of water based on the total weight of the concentrated cleansing composition;
wherein a pH of the concentrated cleansing composition is 3 to 6.

2. The concentrated cleansing composition of claim 1, wherein the anionic surfactant comprises sodium lauroyl glycinate, sodium cocoyl glycinate, sodium lauroyl glutamate, sodium cocoyl glutamate, sodium lauroyl isethionate, sodium cocoyl isethionate, sodium methyl lauroyl taurate, sodium methyl cocoyl taurate, sodium laureth sulfate, sodium pareth sulfate, sodium alpha olefin sulfonate, or a combination thereof.

3. The concentrated cleansing composition of claim 1, wherein the co-surfactant comprises sodium lauroamphoacetate, sodium cocoamphoacetate, sodium lauroamphoacetate, sodium cocoamphoacetate, cocodimethyl carboxymethyl betaine, cocamidopropyl betaine, laurylamidopropyl betaine, cocamidopropyl hydroxysultaine, lauryl hydroxysultaine, cocamide monoethanolamine, or a combination thereof.

4. The concentrated cleansing composition of claim 1, wherein the silicone comprises dimethicone, amodimethicone, cyclomethicone, dimethiconal, dimethiconol and dimethiconol/silsesquioxane copolymer, or a combination thereof.

5. The concentrated cleansing composition of claim 1, wherein the cationic polymer comprises cationic polysaccharide polymers, cationic diallyl quaternary ammonium-containing polymers, mineral acid salts of amino-alkyl esters of homo- and co-polymers of unsaturated carboxylic acids having from 3 to 5 carbon atoms, cationic polyacrylamines, or a combination thereof.

6. The concentrated cleansing composition of claim 1, wherein the cationic polymer comprises cationic cellulose derivatives, cationic starch derivatives, and cationic guar gum deriviatives, dimethyldiallylammonium chloride homopolymer and copolymers of acrylamide and dimethyldiallylammonium chloride, or a combination thereof.

7. The concentrated cleansing composition of claim 1, wherein the cationic polymer comprises guar hydroxypropyltrimethylammonium chloride.

8. The concentrated cleansing composition of claim 1, further comprising a humectant in an amount of less than or equal to 1% by weight based on the total weight of the concentrated cleansing composition.

9. The concentrated cleansing composition of claim 8, wherein the humectant comprises glycerol, propylene glycol, dipropylene glycol, polypropylene glycol, polyethylene glycol, sorbitol, hydroxypropyl sorbitol, hexylene glycol, 1,3-butylene glycol, isoprene glycol, 1,2,6-hexanetriol, ethoxylated glycerol, propoxylated glycerol, or a combination thereof.

10. The concentrated cleansing composition of claim 1, further comprising a hair repair agent in an amount of less than or equal to 3.5% by weight based on the total weight of the concentrated cleansing composition.

11. The concentrated cleansing composition of claim 10, wherein the hair repair agent comprises glucono delta lactone, sodium sulfate, sodium gluconate, trehalose, or a combination thereof.

12. (canceled)

13. (canceled)

14. The concentrated cleansing composition of claim 1, wherein the concentrated cleansing composition has a level of concentration of 2.5 as compared to a base, non-concentrated cleansing composition.

15. The concentrated cleansing composition of claim 1, comprising 4 to 6% by weight based on the total weight of the concentrated cleansing composition of the co-surfactant.

16. The concentrated cleansing composition of claim 2, wherein the anionic surfactant comprises sodium laureth sulfate, sodium pareth sulfate, or a combination thereof.

17. The concentrated cleansing composition of claim 3, wherein the co-surfactant comprises cocamidopropyl betaine, cocamidopropyl hydroxysultaine, lauryl hydroxysultaine, cocamide monoethanolamine, or a combination thereof.

18. The concentrated cleansing composition of claim 9, wherein the humectant comprises glycerol, polyethylene glycol, or a combination thereof.

19. The concentrated cleansing composition of claim 14, wherein the level of concentration is 2.0, as compared to a non-concentrated cleansing composition.
